(19) United States
(12) Patent Application Publication
(10) Pub. No.: US 2020/0197213 A1
(43) Pub. Date: Jun. 25, 2020

(54) FILM FOR AN OSTOMY BAG

(71) Applicant: WELLAND MEDICAL LIMITED, West Sussex (GB)

(72) Inventors: Kim Frampton-Vallance, West Sussex (GB); Mark Newton, West Sussex (GB); Padraig Troy, West Sussex (GB); Barrie Mills, West Sussex (GB)

(73) Assignee: WELLAND MEDICAL LIMITED, West Sussex (GB)

(21) Appl. No.: 16/614,473

(22) PCT Filed: May 24, 2018
(86) PCT No.: PCT/EP2018/063652

§ 371 Date: Nov. 18, 2019

(30) Foreign Application Priority Data
May 25, 2017 (GB) 1708396.5

(51) Int. Cl. A61F 5/445; A61L 28/00; B29C 59/02; B29C 59/06; B29C 59/16; B29C 59/04; B32B 3/30

(52) U.S. Cl. CPC A61F 5/445; A61L 28/0011; B29C 59/022; B32B 2535/00; B29C 59/16; B29C 59/04; B32B 3/30; B29C 59/06

(57) ABSTRACT

A film for an ostomy bag has a first surface and an opposing second surface, the thickness of the film being the distance between each surface, and a plurality of projections are defined in the surfaces to provide a plurality of surface reliefs which are debossed and/or embossed into the film. The film makes minimal noise when one part of the film is moved in relation to another part of the film.

### FIELD OF THE INVENTION

The present invention relates to a film for an ostomy bag. Advantageously, the film makes minimal noise when one part of the film is moved in relation to another part of the film. In addition, the invention is directed to an ostomy bag for collection of waste, wherein the ostomy bag comprises the film.

### BACKGROUND OF THE INVENTION

Ostomy bags are medical devices that are worn by an individual and they can be used for the collection of waste from a surgically diverted bowel or urinary system of the individual. They are used to collect waste that is output from a stoma created in the ostomate's skin and connected to the intestine or urinary system.

Known ostomy bags comprise a pouch, which is also referred to as a collection bag, and, in some cases, the collection bag is attached mechanically or with adhesive to a flange, commonly referred to as a mounting plate, a wafer or a baseplate. The flange is fixed to the skin of an individual and the ostomy bag allows the waste to drain from a stoma into the pouch while protecting the surrounding skin from contamination by the waste.

In general, the pouch comprises an inlet into a waste collection space defined by two opposing surfaces of two walls, the opposing surfaces configured to separate on the introduction of waste through the inlet.

Ostomy bags should be air- and water-tight and they should allow the individual to lead an active normal lifestyle that can include all forms of sport and recreation. However, there is a need to make ostomy bags discreet.

The pouch should be discreet and allow for the ostomy bag to be removed for emptying or disposal. Typically, ostomy bags are emptied at least once per day. However, the need to provide discreet ostomy bags must be balanced with the need to provide a sufficiently large pouch.

One of the problems that users face when using an ostomy bag is that the bag makes a sound during movement of the user.

The experience of wearing an ostomy bag is a necessity in life that ostomates need in order to lead an active normal lifestyle that can include all forms of sport and recreation. As part of this experience, the need to feel comfortable and at ease wearing an ostomy bag is desired. The levels of comfort from wearing an ostomy bag can be determined under a number of methods of perception.

Vision is obviously important and the appearance of an ostomy bag through clothing is preferably minimised. Touch is also important when determining the materials that contact the skin of an ostomate. Surprisingly, the sound of an ostomy bag is an interesting topic of study for researchers and manufacturers alike, as it has been implied that the sounds materials can make from their daily wear may contribute to a sensory experience. In addition, the sounds made by materials used in ostomy bags should be minimised to avoid potential embarrassment by drawing attention to an ostomy bag.

It is known to manufacture pouches for ostomy bags of a waterproof film which commonly has a thickness of about 50 μm to about 100 μm.

The present invention seeks to provide a film for an ostomy bag, wherein the film addresses one or more of the problems presented by prior art arrangements. In this regard, the present invention seeks to provide a film for an ostomy bag which addresses major concerns of ostomates in conformability, comfort, security and discreetness. In particular, the film makes minimal noise when one part of the film is moved in relation to another part of the film.

### SUMMARY OF THE INVENTION

Remarkably, it has now been found that a film for an ostomy bag can be provided which is quiet when the film is manipulated or squeezed into a compact mass. This provides the advantage that noises generally associated with folding or bending of the film are reduced and an ostomy bag can be provided that is more discreet than known ostomy bags.

Accordingly, in a first aspect, the invention provides a film for an ostomy bag wherein the film has a first surface and an opposing second surface, the thickness of the film being the distance between the first and second surfaces, and a plurality of projections are defined in at least one of the surfaces to provide a plurality of surface reliefs which are debossed and/or embossed into the film.

Preferably, each projection defines a peak in one of the surfaces and a corresponding valley in the other surface.

Preferably the projections define a repeating pattern.

Preferably, the surfaces of the film have a pattern of projections defined therein substantially to over the entire surfaces.

Preferably, the cross section and/or base of each projection from where each projection extends from the surface of the film has the shape of a circle, oval, ellipse, square, rectangle, rhombus, lozenge, trapezium, parallelogram, triangle, pentagon, hexagon, heptagon, octagon, nonagon or decagon.

Most preferably, the cross section and/or base of each projection from where each projection extends from the surface of the film has the shape of a hexagon. Remarkably, this shape has been found to be especially effective.

Preferably, the film has a thickness of about 50 μm to about 100 μm, more preferably about 70 μm to about 80 μm, most preferably about 75 μm.

Preferably, each projection defines a cylinder or pocket. Preferably, the height of each projection from the adjoining surface of the film is about 2 mm.

Remarkably, density of the projections appears to be important.

Preferably, each projection is located in a rectangular envelope of a specific size on a surface of the film and each projection abuts the edge of the envelope so that it maximises the space that it occupies in the envelope. In this regard, preferably, each envelope has a width (α) and a length (β). Preferably, α is about 4 mm to about 10 mm. Preferably, β is about 2 mm to about 5 mm.

Preferably, the centre of each envelope is located at a specific distance away from other envelopes. In this regard, the centre of one envelope is preferably a distance x parallel to α and a distance y parallel to β to the centre of an adjacent envelope. Preferably, distance x is about 3 mm to about 6 mm. Preferably, distance y is about 2 mm to about 4 mm.

In a most preferred embodiment, superior results have been found when x is about 3 mm, y is about 2 mm, α is about 4 mm, and β is about 2 mm.

In one embodiment, adjacent projections extend from the film in opposing directions, i.e. from both surfaces of the film.

In an alternative preferred embodiment, projections all extend from the same surface of the film.

Each projection is formed by treatment of a film which comprises the steps of applying one or more of infra-red radiation, forced (hot) air, induction heating, convection heating, selective (localised) heating, compressed air or an air knife/slot. Preferably the projections are formed by one or more of knurled rollers, press (blow moulding), vacuum forming (over a bed or rollers) or by stamping to create an impression.

Preferably, each projection is formed by vacuum forming.

The film is preferably of ethylene vinyl acetate (EVA) or a barrier film laminate having about 5 to about 7 layers, for example a laminate of ethylene vinyl acetate, ethylene vinyl acetate, polyvinylidene chloride, ethylene vinyl acetate, ethylene vinyl acetate (EVA, EVA, PVDC, EVA, EVA) respectively.

The film is preferably selected from a non-laminated film wherein a plurality of projections defined therein or a laminated film wherein each layer of the laminate has a plurality of projections defined therein or at least one layer of the laminated film has a plurality of projections defined therein. A laminated film is selected from a glue laminated film or a non-glue laminated film.

In a second aspect, the invention provides an ostomy bag having a pouch comprising an inlet leading to a waste collection space defined by two opposing surfaces of two walls, the opposing surfaces configured to separate on the introduction of waste through the inlet and including in at least one of the walls a film according to the invention.

Preferably, each projection is not deformable under the weight of waste in the pouch. Advantageously, when the bag fills, no deformation of each projection means that the pouch remains quiet.

Preferably, the walls are welded together at their edges. Preferably, the welding is carried out by impulse welding, heat sealing, ultrasonic welding, radio frequency welding or laser welding.

In an embodiment, the edge of the pouch is defined by an ellipse, having an upper focus, a lower focus, an upper vertex and a lower vertex. Preferably, the inlet leading to a waste collection space is approximately aligned with the upper focus.

Preferably, a flange abuts the inlet. Preferably, the flange is welded to the film. Preferably, the flange comprises a material selected from a material comprising a non-woven material, EVA, polyethylene (e.g. LLDPE or PE or HDPE). Alternatively, the flange is fixed to the film with a flexible or non-flexible adhesive.

Advantageously, despite the projections being formed by vacuum forming, the odour barrier of the bag remains intact.

In one preferred option, the bag comprises a laminate which has hydrophobic exposed surfaces and degradable core and means for delaminating the bag whereby to expose the degradable core and facilitate easy flushing of the bag after use.

In an alternative option, the bag comprises a disposable or degradable liner enclosed in a hydrophobic bag and means for conveniently removing the liner for disposal.

In another option, the bag may further include in an accessible wall a closable exit aperture and means for selectively releasing accumulated gas through the aperture.

The exit aperture can conveniently be resealably opened by a closure which covers the aperture and attaches to the bag by means of a peelable adhesive. The closure may conveniently be provided in the form of a sealing flap which folds down from a seam around the edge of the bag. Alternatively, the closure may comprise a patch which can be completely detached from the bag and optionally replaced with another. Alternative resealable means to a peelable adhesive will no doubt occur to the skilled addressee and include, without limitation, a mechanical seal comprising an annular rib received in an annular groove or a suction based closure means; Velcro® or similar.

In use, when ballooning occurs, the user can discreetly remove the closure, gently squeeze the bag and release gas to return the bag to a comfortable size.

In other options, a valve might be used to close the exit aperture, the valve being easily activated by the user, for example by depressing or nipping an area around the aperture.

### BRIEF DESCRIPTION OF THE DRAWINGS

The invention will now be described by way of example with reference to the accompanying drawings, in which:

FIG. 1 shows the location of projections within envelopes and their location with respect to each other in an embodiment of a film according to the invention. It will be appreciated that projections having cross sections of other shapes not specifically shown also fall within the invention;

FIG. 2A shows a view of two projections in an embodiment according to the invention wherein adjacent projections extend from the film in opposing directions; and FIG. 2B shows a view of two projections in an alternative embodiment according to the invention, wherein adjacent projections extend from the same surface of the film;

FIGS. 3, 4 and 5 show views of various cross sections of projections within envelopes in embodiments of films according to the invention; and

FIG. 6 shows a view of an ostomy bag comprising a film according to the invention together with a magnified section of the film showing projections provided in the surface of the film.

### DETAILED DESCRIPTION OF THE INVENTION

It will be appreciated that aspects, embodiments and preferred features of the invention have been described herein in a way that allows the specification to be written in a clear and concise way. However, unless circumstances clearly dictate otherwise, aspects, embodiments and preferred features can be variously combined or separated in accordance with the invention. In a preferred embodiment, a device in accordance with the invention comprises all aspects of the invention.

Within the context of this specification, the word “about” means preferably plus or minus 20%, more preferably plus or minus 10%, even more preferably plus or minus 5%, most preferably plus or minus 2%.

Within the context of this specification, the word “comprises” means “includes, among other things” and should not be construed to mean “consists of only”.

Within the context of this specification, the word “substantially” means preferably at least 90%, more preferably 95%, even more preferably 98%, most preferably 99%.

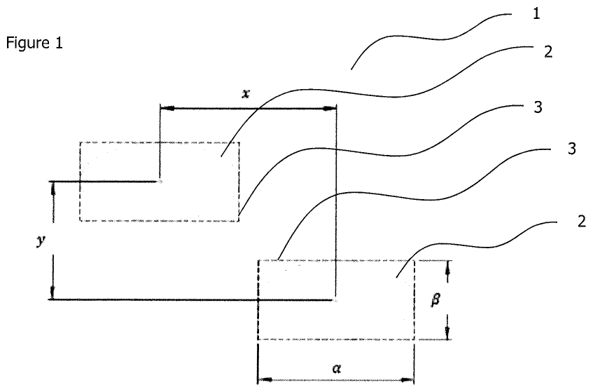

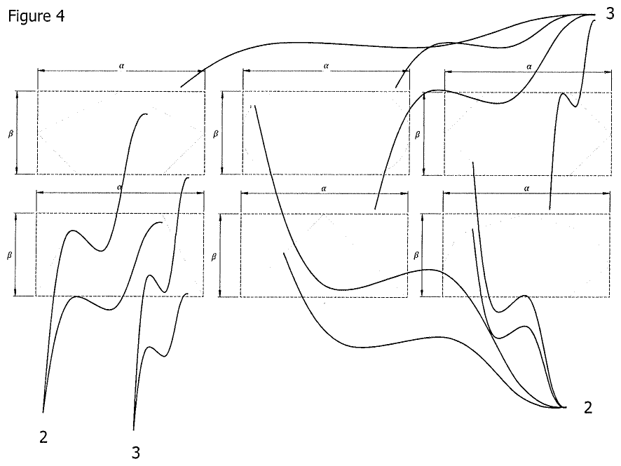

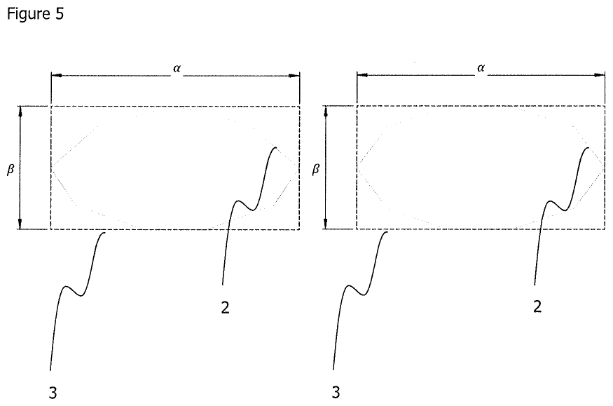

Referring now to FIGS. 1 to 6, FIG. 1 shows the location of projections (2) within envelopes (3) and their location with respect to each other in an embodiment of a film (1) according to the invention. Each projection (2) is located in a rectangular envelope (3) of a specific size on a surface of the film (1) and the corners of each projection (2) abut the edge of the envelope (3). In this regard, each envelope (3) has a width (α) and a length (β). The centre of each envelope (3) is located at a specific distance away from other envelopes (3). In this regard, the centre of one envelope (3) is a distance x parallel to α and a distance y parallel to β to the centre of an adjacent envelope (3).

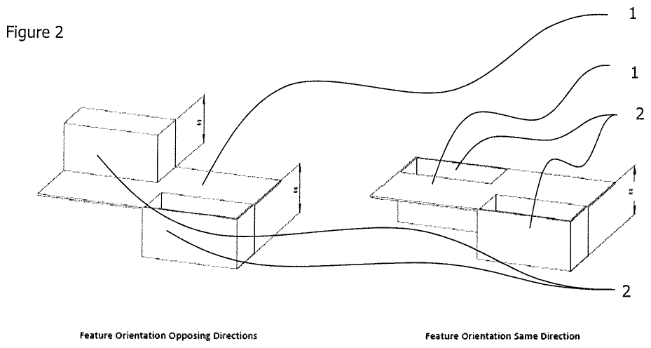

FIG. 2 shows a view of alternative embodiments of films (1), wherein FIG. 2A shows two projections (2) extending from alternative surfaces of the film (1); and FIG. 2B shows two projections (2) extending from the same surface of the film (1). The projections have a height (z).

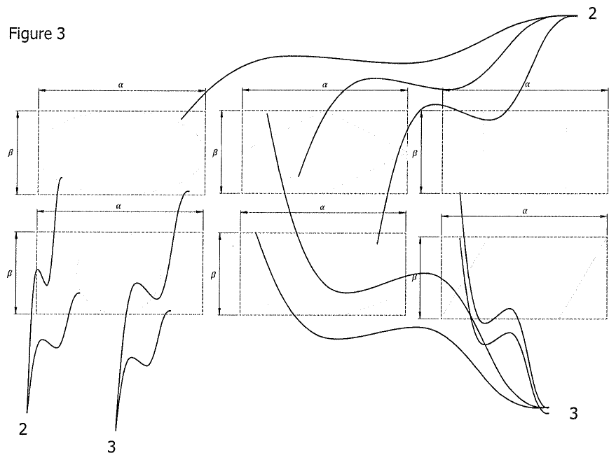

FIGS. 3, 4 and 5 show cross sectional views of alternative embodiments of projections (2) within envelopes (3). Various shape projections in cross sectional view are shown.

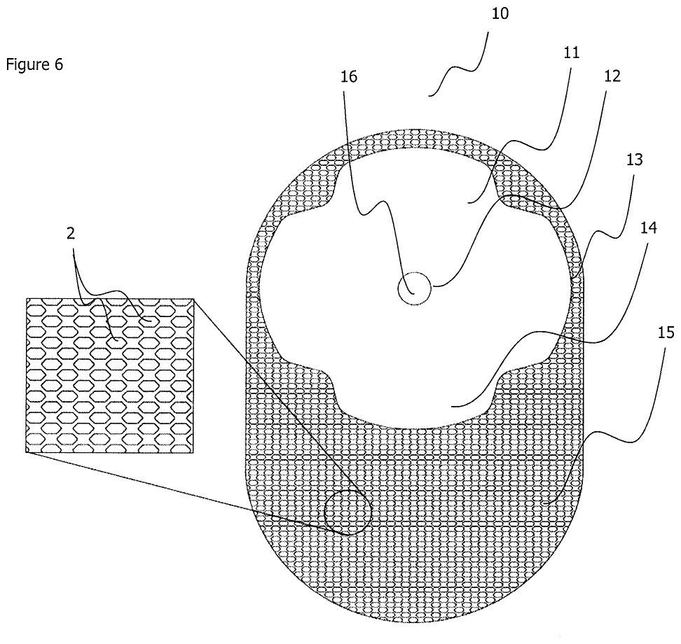

FIG. 6 shows an ostomy bag (10) according to the invention. It has a flange (11) which comprises an annular disk arranged to surround a stomal opening and having a radially inner edge (12) and a radially outer edge (13), the flange (11) having a body facing surface (14) and a pouch facing surface for attachment to a pouch (15).

In an embodiment, the flange (11) is manufactured from a hydrocolloid disc, for example formed of a visco elastic polymer mixed with natural hydrocolloids by heating and pressing and/or by vacuum forming. The flange (11) is provided with a generally central aperture (16) through which the stoma of a user may protrude during use.

The pouch facing surface of the flange typically is bonded to an ostomy appliance such as the pouch (15) of an ostomy bag, for example by means of adhesive or by welding.

In use, the flange (11) with ostomy appliance attached is affixed to the skin of the patient is about the stomal opening, the hydrocolloid adhesive serving to adhere the flange to the skin of the patient. The adhesive seals the flange against the skin. The seal between the flange and the skin serves to prevent waste materials from the stomal opening leaking out of the pouch.

In an embodiment the flange (11) has a uniform thickness of about 0.6 mm to about 1.8 mm, for example 1 mm.

According to the invention, the bag can be a stoma bag, for example an ileostomy, colostomy, or urostomy bag. In addition it can be a closed bag, or a drainable bag having an opening through which the bag may be drained.

### EXAMPLE 1

Test Method and Programme

To test embodiments of the invention after vacuum forming, sound tests were carried out by twisting and scrunching up differing vacuum formed films having a plurality of projections defined in their surfaces and measuring the level of sound generated by the film using a sound meter. Each film tested had projections of varying size, pitch and depth defined in their surfaces.

Details of the various films and projections are described below:

| | Pitch (mm) | | | | |
|---|---|---|---|---|---|
| Sample | x | y | z | α | β |
| 1A | 3 | 2 | 2 | 4 | 2 |
| 1B | 4.5 | 3 | 2 | 4 | 2 |
| 1C | 6 | 4 | 2 | 4 | 2 |
| 1D | 7.5 | 5 | 2 | 4 | 2 |
| 1E | 9 | 6 | 2 | 4 | 2 |
| | 10.5 | 7 | 2 | 4 | 2 |
| | 12 | 8 | 2 | 4 | 2 |
| | 13.5 | 9 | 2 | 4 | 2 |
| | 15 | 10 | 2 | 4 | 2 |
| 1F | 16.5 | 11 | 2 | 4 | 2 |
| | 18 | 12 | 2 | 4 | 2 |
| | 19.5 | 13 | 2 | 4 | 2 |
| 1G | 21 | 14 | 2 | 4 | 2 |
| | 22.5 | 15 | 2 | 4 | 2 |
| | 24 | 16 | 2 | 4 | 2 |
| | 25.5 | 17 | 2 | 4 | 2 |
| | 27 | 18 | 2 | 4 | 2 |
| | 28.5 | 19 | 2 | 4 | 2 |
| | 30 | 20 | 2 | 4 | 2 |
| | 31.5 | 21 | 2 | 4 | 2 |
| | 33 | 22 | 2 | 4 | 2 |
| 1H | 34.5 | 23 | 2 | 4 | 2 |
| | 36 | 24 | 2 | 4 | 2 |
| | 37.5 | 25 | 2 | 4 | 2 |
| 1I | 39 | 26 | 2 | 4 | 2 |
| | 40.5 | 27 | 2 | 4 | 2 |
| | 42 | 28 | 2 | 4 | 2 |
| | 43.5 | 29 | 2 | 4 | 2 |
| | 45 | 30 | 2 | 4 | 2 |
| 1J | 46.5 | 31 | 2 | 4 | 2 |
| | 48 | 32 | 2 | 4 | 2 |
| | 49.5 | 33 | 2 | 4 | 2 |
| 1K | 51 | 34 | 2 | 4 | 2 |
| | 52.5 | 35 | 2 | 4 | 2 |
| | 54 | 36 | 2 | 4 | 2 |
| | 55.5 | 37 | 2 | 4 | 2 |
| | 57 | 38 | 2 | 4 | 2 |
| | 58.5 | 39 | 2 | 4 | 2 |
| | 60 | 40 | 2 | 4 | 2 |
| | 61.5 | 41 | 2 | 4 | 2 |
| | 63 | 42 | 2 | 4 | 2 |
| | 64.5 | 43 | 2 | 4 | 2 |
| | 66 | 44 | 2 | 4 | 2 |
| | 67.5 | 45 | 2 | 4 | 2 |
| | 69 | 46 | 2 | 4 | 2 |
| | 70.5 | 47 | 2 | 4 | 2 |
| | 72 | 48 | 2 | 4 | 2 |
| | 73.5 | 49 | 2 | 4 | 2 |
| | 75 | 50 | 2 | 4 | 2 |
| | 76.5 | 51 | 2 | 4 | 2 |
| | 78 | 52 | 2 | 4 | 2 |
| | 79.5 | 53 | 2 | 4 | 2 |
| | 81 | 54 | 2 | 4 | 2 |
| | 82.5 | 55 | 2 | 4 | 2 |
| | 84 | 56 | 2 | 4 | 2 |
| | 85.5 | 57 | 2 | 4 | 2 |
| | 87 | 58 | 2 | 4 | 2 |
| | 88.5 | 59 | 2 | 4 | 2 |
| | 90 | 60 | 2 | 4 | 2 |
| | 91.5 | 61 | 2 | 4 | 2 |
| | 93 | 62 | 2 | 4 | 2 |
| | 94.5 | 63 | 2 | 4 | 2 |
| | 96 | 64 | 2 | 4 | 2 |
| | 97.5 | 65 | 2 | 4 | 2 |
| | 99 | 66 | 2 | 4 | 2 |
| | 100.5 | 67 | 2 | 4 | 2 |

| | Envelope (mm) | | | | |
|---|---|---|---|---|---|
| Sample | x | y | z | α | β |
| 1A | 3 | 2 | 2 | 4 | 2 |
| 3A | 4.5 | 3 | 2 | 6 | 3 |
| 3B | 6 | 4 | 2 | 8 | 4 |
| 3C | 7.5 | 5 | 2 | 10 | 5 |
| 3D | 9 | 6 | 2 | 12 | 6 |
| 3E | 10.5 | 7 | 2 | 14 | 7 |
| 3F | 12 | 8 | 2 | 16 | 8 |
| 3G | 13.5 | 9 | 2 | 18 | 9 |
| 3H | 15 | 10 | 2 | 20 | 10 |
| 3I | 16.5 | 11 | 2 | 22 | 11 |
| 3J | 18 | 12 | 2 | 24 | 12 |
| 3K | 19.5 | 13 | 2 | 26 | 13 |
| 3L | 21 | 14 | 2 | 28 | 14 |
| | 22.5 | 15 | 2 | 30 | 15 |
| | 24 | 16 | 2 | 32 | 16 |
| | 25.5 | 17 | 2 | 34 | 17 |
| | 27 | 18 | 2 | 36 | 16 |
| | 28.5 | 19 | 2 | 38 | 19 |
| | 30 | 20 | 2 | 40 | 20 |
| | 31.5 | 21 | 2 | 42 | 21 |
| | 33 | 22 | 2 | 44 | 22 |
| | 34.5 | 23 | 2 | 46 | 23 |
| | 36 | 24 | 2 | 48 | 24 |
| | 37.5 | 25 | 2 | 50 | 25 |
| | 39 | 26 | 2 | 52 | 26 |
| | 40.5 | 27 | 2 | 54 | 27 |
| | 42 | 26 | 2 | 56 | 28 |
| | 43.5 | 29 | 2 | 58 | 29 |
| | 45 | 30 | 2 | 60 | 30 |
| | 46.5 | 31 | 2 | 62 | 31 |
| | 48 | 32 | 2 | 64 | 32 |
| | 49.5 | 33 | 2 | 66 | 33 |
| | 51 | 34 | 2 | 68 | 34 |
| | 52.5 | 35 | 2 | 70 | 35 |
| | 54 | 36 | 2 | 72 | 36 |
| | 55.5 | 37 | 2 | 74 | 37 |
| | 57 | 38 | 2 | 76 | 38 |
| | 58.5 | 39 | 2 | 78 | 39 |
| | 60 | 40 | 2 | 80 | 40 |
| | 61.5 | 41 | 2 | 82 | 41 |
| | 63 | 42 | 2 | 84 | 42 |
| | 64.5 | 43 | 2 | 86 | 43 |
| | 66 | 44 | 2 | 88 | 44 |
| | 67.5 | 45 | 2 | 90 | 45 |
| | 69 | 46 | 2 | 92 | 46 |
| | 70.5 | 47 | 2 | 94 | 47 |
| | 72 | 48 | 2 | 96 | 48 |
| | 73.5 | 49 | 2 | 98 | 49 |
| | 75 | 50 | 2 | 100 | 50 |
| | 76.5 | 51 | 2 | 102 | 51 |
| | 78 | 52 | 2 | 104 | 52 |
| | 79.5 | 53 | 2 | 106 | 53 |
| | 81 | 54 | 2 | 108 | 54 |
| | 82.5 | 55 | 2 | 110 | 55 |
| | 84 | 56 | 2 | 112 | 56 |
| | 85.5 | 57 | 2 | 114 | 57 |
| | 87 | 58 | 2 | 116 | 58 |
| | 88.5 | 59 | 2 | 118 | 59 |
| | 90 | 60 | 2 | 120 | 60 |
| | 91.5 | 61 | 2 | 122 | 61 |
| | 93 | 62 | 2 | 124 | 62 |
| | 94.5 | 63 | 2 | 126 | 63 |
| | 96 | 64 | 2 | 128 | 64 |
| | 97.5 | 65 | 2 | 130 | 65 |
| | 99 | 66 | 2 | 132 | 66 |
| | 100.5 | 67 | 2 | 134 | 67 |

Optimal Envelope/Pitch Dimensions

Test Results

The results indicate that vacuum forming of the films tested to a draw depth of 2 mm for the shaded pitch and envelope sizes achieved remarkably superior results compared to films tested to a greater draw depth and with greater pitch and envelope sizes.

### EXAMPLE 2

Further comparative tests were carried out as described below.

1. Summary

During comparative tests, a hexagonal prism configuration (1A/A1) being utilised for the construction of a film according to the invention averaged a value of 36.30 dB, making it comparable to a commercially available pouch.

2. Procedure

Samples were tested in a reverberation room (constructed from 215 mm brick that is internally plastered with a reinforced concrete floor and roof. The room had a volume of 300 m³ and was isolated by the use of resilient mountings and seals from the surrounding structure ensuring good acoustic isolation), and rustled at approximately one revolution per second.

Samples were rustled and once a consistent noise level was achieved, a measurement was taken for ten seconds, at a distance of one metre.

The samples were tested in accordance with BS EN ISO 3741:2010 “Determination of sound power levels and sound energy levels of noise sources using sound pressure—Precision Methods for reverberation test rooms”. In accordance with these tests, the sound power level or sound energy level of a noise source from sound pressure levels was measured in a reverberation test room. The sound power level (or, in the case of noise bursts or transient noise emission, the sound energy level) produced by the noise source, in frequency bands of width one-third-octave, is calculated using those measurements, including corrections to allow for any differences between the meteorological conditions at the time and place of the test and those corresponding to a reference characteristic impedance. Measurement and calculation procedures are given for both a direct method and a comparison method of determining the sound power level and the sound energy level.

In general, the frequency range of interest includes the one-third-octave bands with mid-band frequencies from 100 Hz to 10 000 Hz. The tests were not applicable to frequency ranges above the 10 000 Hz one-third-octave band.

The methods specified were suitable for all types of noise (steady, non-steady, fluctuating, isolated bursts of sound energy, etc.).

The noise source under test can be a device, machine, component or sub-assembly. The test are applicable to noise sources with a volume not greater than 2% of the volume of the reverberation test room. For a source with a volume greater than 2% of the volume of the test room, it is possible that the achievement of precise results is not feasible.

In accordance with the tests, a pole having a length of one meter was positioned between a sound level meter and the sample to be tested. The sound level meter was positioned on a first tripod, the pole was positioned on a second tripod and the sample was suspended adjacent an end of the pole distal to the sound level meter. This ensured that the sample was held at a consistent distance from the sound level meter.

3. Results

The comparative test results are shown in the table below:

| | | | | | | | | | | | Average | |
|---|---|---|---|---|---|---|---|---|---|---|---|---|
| | Description | | | | | | | | Lp | Lp | (dB & | |
| Sample | Type | x | | y | z | α | | β | (dB) | (dBA) | dBA) | Rank |
| X1 | Commercially | n/a | | n/a | n/a | n/a | | n/a | 44.60 | 44.80 | 44.70 | 11 |
| | Available A | | | | | | | | | | | |
| X2 | Commercially | n/a | | n/a | n/a | n/a | | n/a | 34.90 | 33.80 | 34.35 | 2 |
| | Available B | | | | | | | | | | | |
| X3 | Commercially | n/a | | n/a | n/a | n/a | | n/a | 42.80 | 42.40 | 42.60 | 10 |
| | Available C | | | | | | | | | | | |
| A | Plain film having | n/a | | n/a | n/a | n/a | | n/a | 47.50 | 47.40 | 47.45 | 14 |
| | thickness of 63 um | | | | | | | | | | | |
| B | Plain film having | n/a | | n/a | n/a | n/a | | n/a | 47.60 | 47.70 | 47.65 | 15 |
| | thickness of 75 um | | | | | | | | | | | |
| C | Plain film having | n/a | | n/a | n/a | n/a | | n/a | 49.00 | 49.20 | 49.10 | 16 |
| | thickness of 100 um | | | | | | | | | | | |
| A1 | Embodiment of | 3 | mm | 2 mm | 2 mm | 4 | mm | 2 mm | 38.40 | 32.70 | 35.55 | 3 |
| | the invention | | | | | | | | | | | |
| 1A | Embodiment of | 3 | mm | 2 mm | 2 mm | 4 | mm | 2 mm | 38.90 | 35.20 | 37.05 | 5 |
| | the invention | | | | | | | | | | | |
| 1B | Embodiment of | 4.5 | mm | 3 mm | 2 mm | 4 | mm | 2 mm | 47.40 | 46.20 | 46.80 | 13 |
| | the invention | | | | | | | | | | | |
| 1C | Embodiment of | 6 | mm | 4 mm | 2 mm | 4 | mm | 2 mm | 46.00 | 45.30 | 45.65 | 12 |
| | the invention | | | | | | | | | | | |
| 3A | Embodiment of | 4.5 | mm | 3 mm | 2 mm | 6 | mm | 3 mm | 43.70 | 41.40 | 42.55 | 9 |
| | the invention | | | | | | | | | | | |
| 3B | Embodiment of | 6 | mm | 4 mm | 2 mm | 8 | mm | 4 mm | 35.90 | 30.40 | 33.15 | 1 |
| | the invention | | | | | | | | | | | |
| 3C | Embodiment of | 7.5 | mm | 5 mm | 2 mm | 10 | mm | 5 mm | 40.40 | 34.70 | 37.55 | 7 |
| | the invention | | | | | | | | | | | |
| 3D | Embodiment of | 9 | mm | 6 mm | 2 mm | 12 | mm | 6 mm | 38.30 | 34.60 | 36.45 | 4 |
| | the invention | | | | | | | | | | | |
| 3E | Embodiment of | 10.5 | mm | 7 mm | 2 mm | 14 | mm | 7 mm | 44.00 | 40.20 | 42.10 | 8 |
| | the invention | | | | | | | | | | | |
| 3F | Embodiment of | 12 | mm | 8 mm | 2 mm | 16 | mm | 8 mm | 44.10 | 40.10 | 42.10 | 8 |
| | the invention | | | | | | | | | | | |
| 3J | Embodiment of | 18 | mm | 12 mm | 2 mm | 24 | mm | 12 mm | 38.70 | 36.20 | 37.45 | 6 |
| | the invention | | | | | | | | | | | |
| 3L | Embodiment of | 21 | mm | 14 mm | 2 mm | 28 | mm | 14 mm | 42.90 | 42.20 | 42.55 | 9 |
| | the invention | | | | | | | | | | | |

4. Conclusion

Based upon the results obtained in the comparative tests, the majority of the formed films of the invention demonstrated a lower dB level in the tests compared to films of standard manufactured ostomy pouches providing evidence that films of the invention can be used to manufacture quieter ostomy pouches.

The above described embodiments have been given by way of example only, and the skilled reader will naturally appreciate that many variations could be made thereto without departing from the scope of the claims.

1. A film for an ostomy bag wherein the film has a first surface and an opposing second surface, the thickness of the film being the distance between the first and second surfaces, and a plurality of projections are defined in at least one of the surfaces to provide a plurality of surface reliefs which are debossed and/or embossed into the film.

2. A film according to claim 1, wherein each projection defines a peak in one of the first or second surfaces and a corresponding valley in the other surface.

3. A film according to claim 1, wherein the projections are spaced apart from each other to define a repeating pattern.

4. A film according to claim 1, wherein the first and second surfaces of the film have a pattern of projections defined therein substantially over the entire surfaces.

5. A film according to claim 1, wherein each projection has a base from where each projection extends from the surface of the film and a cross section parallel to the base, wherein the base and/or the cross section has the shape of a circle, oval, ellipse, square, rectangle, rhombus, lozenge, trapezium, parallelogram, triangle, pentagon, hexagon, heptagon, octagon, nonagon or decagon.

6. A film according to claim 5, wherein each projection has a base from where each projection extends from the surface of the film and a cross section parallel to the base, wherein the base and/or cross section have the shape of a hexagon.

7. A film according to claim 1, wherein the film has a thickness of about 50 μm to about 100 μm.

8. A film according to claim 1, wherein each projection defines a cylinder or pocket.

9. A film according to claim 1, wherein each projection has a height from the adjoining surface of the film of about 2 mm.

10. A film according to claim 1, wherein each projection is located in a rectangular envelope having a width (α) and a length (β) on a surface of the film and each projection abuts the edge of the envelope so that it maximises the space that it occupies in the envelope

11. A film according to claim 10, wherein α is about 4 mm to about 10 mm

12. A film according to claim 10 wherein β is about 2 mm to about 5 mm.

13. A film according to claim 10 wherein the centre of each envelope is located a distance x parallel to α and a distance y parallel to β to the centre of an adjacent envelope.

14. A film according to claim 13, wherein distance x is about 3 mm to about 6 mm.

15. A film according to claim 13, wherein distance y is about 2 mm to about 4 mm.

16. A film according to claim 13, wherein x is about 3 mm or about 6 mm, y is about 2 mm or about 4 mm, α is about 4 mm or about 8 mm, and β is about 2 mm or about 4 mm.

17. A film according to claim 1, wherein adjacent projections extend from the film in opposing directions, i.e. from both surfaces of the film.

18. A film according to claim 1, wherein adjacent projections extend from the film in the same direction, i.e. from the same surface of the film.

19. A film according to claim 1, wherein each projection is formed by treatment of a film which comprises the steps of applying one or more of infra-red radiation, forced (hot) air, induction heating, convection heating, selective (localised) heating, compressed air or an air knife/slot.

20. A film according to claim 1, wherein the projections are formed by one or more of knurled rollers, press (blow moulding), vacuum forming (over a bed or rollers) or by stamping to create an impression.

21. A film according to claim 1, wherein the film is preferably of ethylene vinyl acetate (EVA) or a barrier film laminate having about 5 to about 7 layers, for example a laminate of ethylene vinyl acetate, ethylene vinyl acetate, polyvinylidene chloride, ethylene vinyl acetate, ethylene vinyl acetate (EVA, EVA, PVDC, EVA, EVA) respectively.

22. A film according to claim 1, wherein the film is preferably selected from a non-laminated film wherein a plurality of projections defined therein or a laminated film wherein each layer of the laminate has a plurality of projections defined therein or at least one layer of the laminated film has a plurality of projections defined therein.

23. A film according to claim 22, wherein a laminated film is selected from a glue laminated film or a non-glue laminated film.

24. An ostomy bag having a pouch comprising an inlet leading to a waste collection space defined by two opposing surfaces of two walls, the opposing surfaces configured to separate on the introduction of waste through the inlet and including in at least one of the walls a film according to claim 1.

25. An ostomy bag according to claim 24, wherein the walls are welded together at their edges.

26. An ostomy bag according to claim 24, wherein a flange abuts the inlet.

27. (canceled)
